(19) United States
(12) Patent Application Publication
(10) Pub. No.: US 2022/0087581 A1
(43) Pub. Date: Mar. 24, 2022

(54) Blood Collection Devices, Systems, and Methods to Facilitate Air Priming

(71) Applicant: Becton, Dickinson and Company, Franklin Lakes, NJ (US)

(72) Inventor: Tao Jiang, Suzhou Jiangsu (CN)

(21) Appl. No.: 17/479,500

(22) Filed: Sep. 20, 2021

Related U.S. Application Data
(60) Provisional application No. 63/081,091, filed on Sep. 21, 2020.

(51) Int. Cl. A61B 5/15

(52) U.S. Cl. CPC A61B 5/150213; A61B 5/150244

(57) ABSTRACT

A blood collection device to prime a blood flow path may include a cannula, a holder, and an elastomeric sleeve. The cannula may include a proximal end, a distal end, and a lumen extending along a longitudinal axis therebetween. The cannula may include a slot to enable a user to visualize the blood flow path through the cannula. The holder may include a channel configured to align with the longitudinal axis to retain a shaft of the cannula. The holder may further include a visual check window corresponding to the slot of the cannula. The elastomeric sleeve may be coupled to the holder and enclose the proximal end of the cannula. A priming opening may be disposed between the elastomeric sleeve and the holder to prime the blood flow path.

### CROSS-REFERENCE TO RELATED APPLICATION

The present application claims priority to U.S. Provisional Application Ser. No. 63/081,091, entitled “Blood Collection Devices, Systems, and Methods to Facilitate Air Priming”, filed Sep. 21, 2020, the entire disclosure of which is hereby incorporated by reference in its entirety.

### BACKGROUND OF THE INVENTION

Intravenous catheters are commonly used for a variety of infusion therapies. For example, intravenous catheters may be used for infusing fluids, such as normal saline solution, various medicaments, and total parenteral nutrition, into a patient. Intravenous catheters may also be used for withdrawing blood from the patient.

Common types of intravenous catheter are peripheral IV catheters (“PIVCs”), peripherally inserted central catheters (“PICCs”), and midline catheters. Intravenous catheters may include “over-the needle” catheters, which may be mounted over a needle having a sharp distal tip. The sharp distal tip may be used to pierce skin and the vasculature of the patient. Insertion of the intravenous catheter into the vasculature may follow the piercing of the vasculature by the needle. The needle and the intravenous catheter are generally inserted at a shallow angle through the skin into the vasculature of the patient with a bevel of the needle facing up and away from the skin of the patient.

In order to verify proper placement of the introducer needle and/or the intravenous catheter in the vasculature, a user generally confirms that there is flashback of blood, which may be visible to the user. In some instances, the introducer needle may include a notch disposed towards a distal end of the introducer needle, and in response to the distal tip of the introducer needle being positioned within the vasculature, blood may flow proximally through a needle lumen, exit the needle lumen through the notch, and then travel proximally between an outer surface of the introducer needle and an inner surface of the intravenous catheter.

Accordingly, where the intravenous catheter is at least partially transparent, the user may visualize a small amount of blood “flashback” and thereby confirm placement of the intravenous catheter within the vasculature. Presence of a vasculature entrance indicator, such as flashback, may facilitate successful placement of intravenous catheters. Flashback may also be used to conduct air priming to purge air out of the flow path. Once placement of the introducer needle within the vasculature has been confirmed, the user may temporarily occlude flow in the vasculature and withdraw the introducer needle, leaving the intravenous catheter in place for future blood withdrawal and/or fluid infusion.

For blood withdrawal, a blood collection tube may be attached to the catheter via a connector. A VACUTAINER™ blood collection tube, for example, may include a sterile glass or plastic test tube with a rubber stopper that creates a vacuum inside of the tube, facilitating the drawing of a predetermined volume of blood from a patient. In order to draw blood from the patient, a double-ended needle may be used. A proximal end of the needle may be positioned inside a holder, and a distal end of the needle may be inserted into the catheter or a vein of a patient. When the VACUTAINER™ blood collection tube is inserted into the holder, the rubber stopper may be punctured by the proximal end of the needle, and the vacuum in the tube may pull blood through the needle into the tube. The filled tube may then be removed and another tube can be inserted and filled the same way.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure generally relates to blood collection devices, systems, and methods to prime a blood flow path. In some embodiments, a blood collection device may include a cannula, a holder, and an elastomeric sleeve. In some embodiments, the cannula may include a proximal end, a distal end, and a lumen extending along a longitudinal axis therebetween. Some embodiments of the cannula may include a slot to enable a user to visualize a blood flow path through the cannula.

In some embodiments, the holder may include a proximal end, a distal end, and a channel extending therebetween. Some embodiments of the channel may be configured to align with the longitudinal axis to retain a shaft of the cannula. In some embodiments, the holder may include a visual check window corresponding to the slot of the cannula.

In some embodiments, the elastomeric sleeve may be coupled to the holder and may enclose the proximal end of the cannula. In some embodiments, a priming opening may be disposed between the elastomeric sleeve and the holder to prime the blood flow path. In some embodiments, the priming opening may be automatically sealed in response to a blood collection tube being coupled to the holder. In some embodiments, the elastomeric sleeve may automatically seal the priming opening.

In some embodiments, the holder may be configured to couple to the blood collection tube such that the proximal end of the cannula and the elastomeric sleeve may extend through a distal end of the blood collection tube. In some embodiments, the holder may be configured to couple to a needleless connector. In some embodiments, the holder may include a contact interface for physical manipulation of the cannula.

In some embodiments, the holder may include a projection extending from the proximal end of the holder along the longitudinal axis. Some embodiments of the projection may include at least a portion of the channel. In some embodiments, the projection may include the priming opening.

Some embodiments of a blood collection system may include an adapter such as a Y-adapter comprising a distal end, a first proximal port, and a second proximal port. In some embodiments, the blood collection system may further include a blood collection device coupled to the first proximal port.

In some embodiments, the blood collection device may include a body including a distal end, a proximal end, and a longitudinal axis extending therebetween. In some embodiments, a septum may be disposed within the body. In some embodiments, a cannula having a distal end and a sharp proximal end may extend through the septum. In some embodiments, an elastomeric sleeve may be coupled to the body and cover the sharp proximal end of the cannula.

In some embodiments, an air pipe may extend through the septum. In some embodiments, an air membrane may be disposed proximal to the air pipe. Some embodiments of an air priming pathway may include the air pipe and the air membrane. In some embodiments, a blood collection pathway separate from the air priming pathway includes the cannula.

In some embodiments, the distal end of the body may include a male luer adapter, and the cannula may extend through the proximal end of the body. In some embodiments, the body may include an extension between the septum and the elastomeric sleeve. Some embodiments of the extension may be configured to be held by a user. In some embodiments, the air membrane may be disposed within the extension.

In some embodiments, the elastomeric sleeve may automatically seal the air priming pathway in response to a blood collection tube being coupled to the body. In some embodiments, the blood collection system may further include a seal ring disposed within the first proximal port such that the cannula extends through the seal ring. In some embodiments, the seal ring may be disposed at a distal end of the first proximal port. In some embodiments, the seal ring may be disposed adjacent to the septum.

In some embodiments, the air pipe may include multiple channels. Some embodiments of the blood collection pathway may include a first channel and some embodiments of the air priming pathway may include a second channel. In some embodiments, the air priming pathway may include an air rod to replace the air pipe and the air membrane. Some embodiments of the air rod may include a porous material to allow air therethrough and to block liquid. In some embodiments, the air rod may be integrated with the body of the blood collection device.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of a blood collection system implementing a Y-adapter, according to some embodiments;

FIG. 1B is an upper perspective view of another example blood collection system implementing a Y-adapter, according to some embodiments;

FIG. 2A is a side perspective view of a blood collection device used in connection with a Y-adapter, according to some embodiments;

FIG. 2B is another side perspective view of the blood collection device of FIG. 2A used in connection with the Y-adapter of FIG. 2A, according to some embodiments;

FIG. 2C is an upper perspective view of the blood collection device of FIG. 2A used in connection with the Y-adapter of FIG. 2A, according to some embodiments;

FIG. 3 is a perspective view of multiple blood collection devices, according to some embodiments;

FIG. 4A is a perspective view of one of the blood collection devices of FIG. 3 integrated with an example catheter, according to some embodiments;

FIG. 4B is a perspective view of one of the blood collection devices of FIG. 3 integrated with an example catheter assembly, according to some embodiments;

FIG. 4C is an upper cross-sectional view of one of the blood collection devices of FIG. 3 integrated with an example catheter adapter, according to some embodiments;

FIG. 5 is a cross-sectional view of an example blood collection device, according to some embodiments;

FIG. 6 is a perspective view of the blood collection device of FIG. 5, according to some embodiments;

FIG. 7 is a cross-sectional view of another example blood collection device, according to some embodiments;

FIG. 8 is a perspective view of the blood collection device of FIG. 7, according to some embodiments;

FIG. 9A is a perspective view of an example holder, according to some embodiments;

FIG. 9B is a cross-sectional view of another example catheter system, according to some embodiments;

FIG. 10A is a perspective view of another example holder, according to some embodiments;

FIG. 10B is a perspective view of an example blood collection device incorporating the holder of FIG. 10A, according to some embodiments;

FIG. 11A is a perspective view of another example holder, according to some embodiments;

FIG. 11B is a perspective view of an example blood collection device incorporating the holder of FIG. 11A, according to some embodiments;

FIG. 12A is a perspective view of another example holder, according to some embodiments;

FIG. 12B is a perspective view of an example blood collection device incorporating the holder of FIG. 12A, according to some embodiments;

FIG. 13A is a perspective view of another example holder, according to some embodiments;

FIG. 13B is a perspective view of an example blood collection device incorporating the holder of FIG. 13A, according to some embodiments;

FIG. 14 is a cutaway perspective view of an example blood collection device integrated with a Y-adapter, according to some embodiments;

FIG. 15 is a cross-sectional perspective view of the example blood collection device of FIG. 14, according to some embodiments;

FIG. 16A is a perspective view of an example seal ring, according to some embodiments;

FIG. 16B is a perspective view of another example seal ring, according to some embodiments;

FIG. 17A is a cutaway perspective view of an example blood collection device illustrating the seal ring of FIG. 16B, according to some embodiments;

FIG. 17B is a cutaway perspective view of another example blood collection device illustrating the seal ring of FIG. 16A, according to some embodiments;

FIG. 18A is a cutaway perspective view of an example blood collection device illustrating an air pipe having multiple channels, according to some embodiments;

FIG. 18B is an enlarged view of a portion of the blood collection device and air pipe of FIG. 18A, according to some embodiments;

FIG. 19A is a cutaway perspective view of an example blood collection device illustrating an example air pipe, according to some embodiments; and

FIG. 19B is a cutaway perspective view of an example blood collection device illustrating an example air rod, according to some embodiments.

### DESCRIPTION OF EMBODIMENTS

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

Referring now to Figures lA and 1B, in some embodiments, a blood collection system 10 may communicate with a catheter assembly 2 via an adapter 14, such as a Y-adapter. In some embodiments, a catheter 4 of the catheter assembly 2 may be disposed within a vasculature of a patient for infusion of saline or medicaments, as well as for blood withdrawal.

In some embodiments, medical liquid or saline may be infused through a side port 6 of the adapter 14 to purge air out of the flow path prior to insertion of the catheter 4 into the vasculature. Connecting a vacuum blood collection tube 50 to the adapter 14, however, may cause the medical liquid or saline to mix with the blood sample, thereby diluting the blood sample and potentially causing inaccurate diagnoses. Some clinicians utilize blood flashback to purge air from the flow path. However, blood may congeal inside the adapter 14 after blood sampling if the air priming procedure is not conducted properly or adequately. Embodiments disclosed herein address these and other issues.

Some embodiments of the blood collection system 10 may include the adapter 14 comprising a distal port 58, a first proximal port opening 60, and a second proximal port 62. In some embodiments, the adapter 14 may include a Y-adapter, a T-adapter, or other suitable adapter 14. In some embodiments, a blood collection device 12 may be coupled to the first proximal port 60 via a needless adapter 54 such as a Luer adapter, for example.

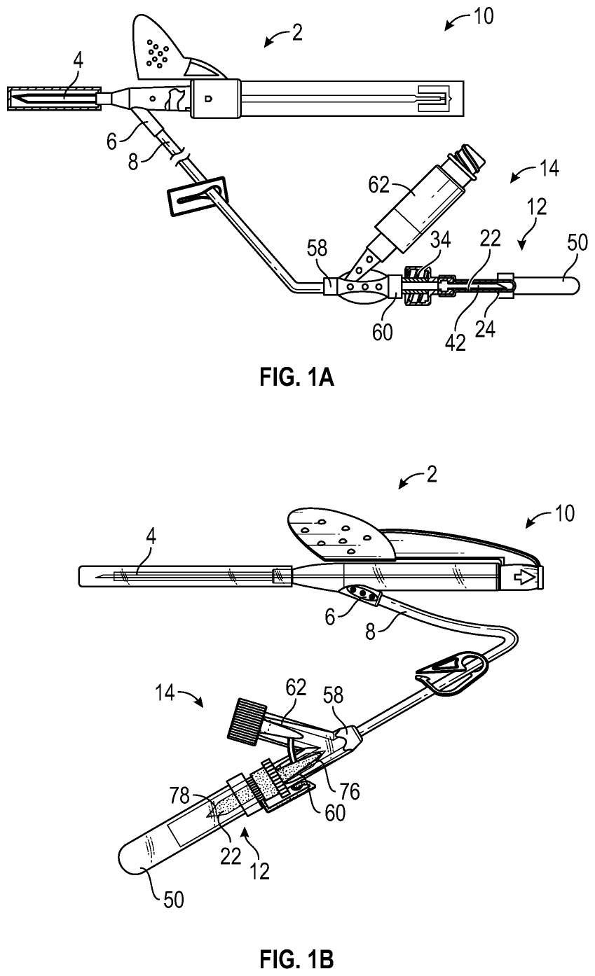

Referring now to FIG. 1A, in some embodiments, the blood collection device 12 may include a cannula 22 having a slot 32 to enable a user to visualize a blood flow path through the cannula 22. In some embodiments, a shaft 42 of the cannula 22 may be retained by holder 34. In some embodiments, the cannula 22 may be made of steel or another suitable biocompatible material. In some embodiments, the holder 34 may be made of a polymer, rubber, or another suitable material. Some embodiments of the holder 34 may include a visual check window 44 corresponding to the slot 32 of the cannula 22.

In some embodiments, an elastomeric sleeve 46 may be coupled to the holder 34 and may enclose the proximal end 24 of the cannula 22. In some embodiments, a priming opening 48 may be disposed between the elastomeric sleeve 46 and the holder 34 to prime the blood flow path. In some embodiments, the priming opening 48 may be automatically sealed in response to a blood collection tube 50 being coupled to the holder 34.

Referring now to FIG. 1B, in some embodiments, the blood collection device 12 may include a body 64 having a septum 72 disposed therein. In some embodiments, a cannula 22 having a distal end 76 and a sharp proximal end 78 may extend through the septum 72. In some embodiments, an elastomeric sleeve 46 may be coupled to the body 64 to cover the sharp proximal end 78 of the cannula 22. In some embodiments, an air priming pathway may be separate from a blood collection pathway to prime the blood collection device 12. In some embodiments, the elastomeric sleeve 46 may automatically seal the air priming pathway in response to a blood collection tube 50 being coupled to the body 64.

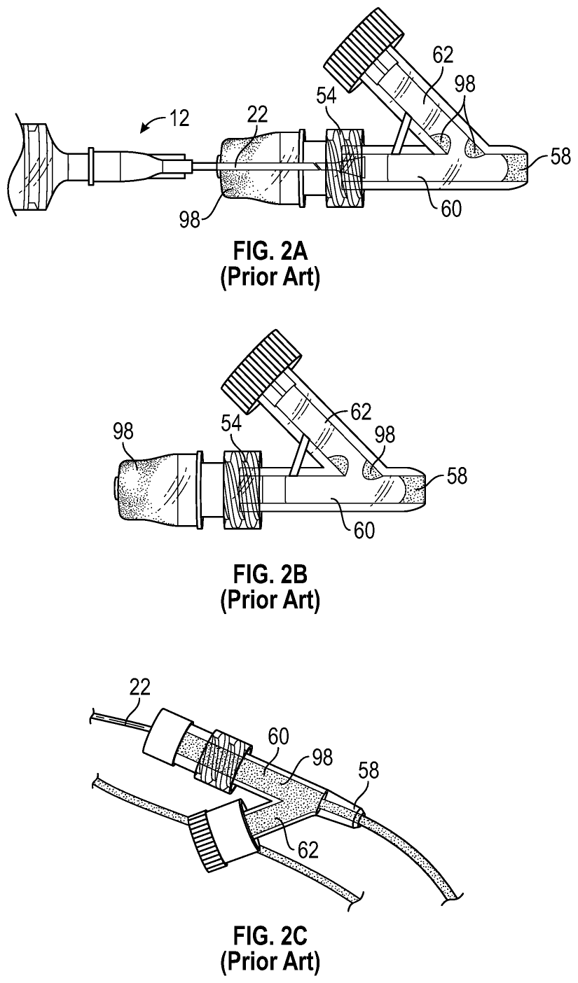

Referring now to FIGS. 2A, 2B, and 2C, in some prior art blood collection devices 12, there is a risk of contamination from residual blood 98 inside the adapter 14 and/or needleless adapter 54 after blood sampling. Indeed, if the air priming procedure is not conducted properly or adequately residual blood 98 may contaminate infusates or blood samples obtained after the air priming procedure.

As shown in FIGS. 2A and 2B, in prior art blood collection devices 12, it is common for residual blood 98 to accumulate along the inside walls of the adapter 14, usually at or near a juncture between the first proximal port 60 and the second proximal port 62 or at the distal port 58. It is also common for residual blood 98 to accumulate along or within threads or crevices of the needleless adapter 54, or along surfaces of a cap 100 attached to the needleless adapter 54. In some cases, as shown in FIG. 2C, blood 98 may congeal within the adapter 14 and/or catheter or tubing attached thereto. This may occlude the fluid pathway in addition to contaminating infusates or blood samples obtained after the air priming procedure.

In other cases, prior art blood collection devices 12 may not enable a user to visualize blood flashback absent a blood collection tube 50 attached thereto. In other cases, some prior art blood collection devices 12 may not allow for performance of air priming procedures at all, which may adversely affect a volume of a first sample.

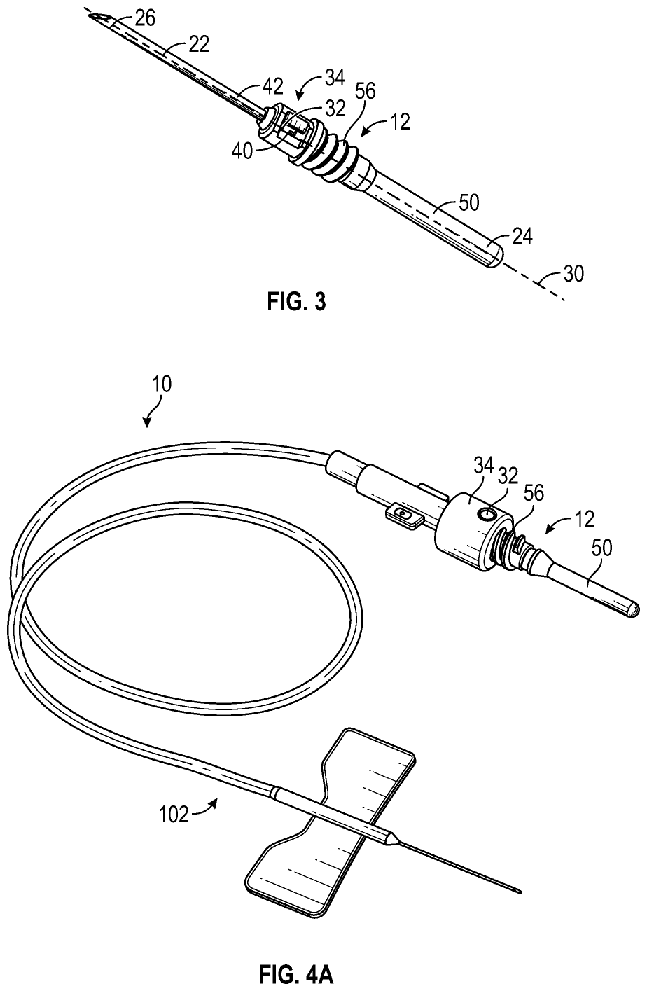

Referring now to FIG. 3, some embodiments of blood collection devices 12 disclosed and claimed herein may address the risks and disadvantages of prior art devices set forth above. As shown in FIG. 3, in some embodiments, the blood collection device 12 may include the cannula 22, the holder 34, and the elastomeric sleeve 46. In some embodiments, the cannula 22 may include a proximal end 24, a distal end 26, and a lumen 20 therebetween. In some embodiments, the lumen 20 may extend along a longitudinal axis 30.

Some embodiments of the holder 34 may enable a user to hold and physically manipulate the cannula 22. In some embodiments, the holder 34 may include a contact interface 56 for physical manipulation of the cannula 22. In some embodiments, the holder 34 may include a proximal end 36, a distal end 38, and a channel 40 extending therebetween. Some embodiments of the channel 40 may retain a shaft 42 of the cannula 22.

Some embodiments of the cannula 52 may include a slot 32 to enable a user to visualize a blood flow path through the cannula 22. Similarly, some embodiments of the holder 34 may include a visual check window 44 corresponding to the slot 32 of the cannula 22. Some embodiments of the blood collection device 12 may be configured such that the slot 32 aligns with the visual check window 454, thus enabling a user to visualize the blood flow path or blood flashback through the blood collection device 12.

In some embodiments, an elastomeric sleeve 46 may be coupled to the holder 34 and may enclose the proximal end 24 of the cannula 22. Some embodiments of the elastomeric sleeve 46 may include rubber or another suitable elastomeric material. In some embodiments, the elastomeric sleeve 46 may be biocompatible and may be substantially liquid impermeable.

In some embodiments, one or more priming openings 48 may be disposed between the elastomeric sleeve 46 and the holder 34 and may be proximate to an interface therebetween. In some embodiments, the elastomeric sleeve 46 may act as a trigger for priming and blood sampling. For example, upon insertion of the blood collection device 12 into the vasculature of the patient, the elastomeric sleeve 46 may prime air present in the blood flow path through the priming opening 48. Some embodiments of the priming opening 48 may include a clearance opening 54 and a priming slot 55. In some embodiments, blood present inside the blood flow path may be retained by the elastomeric sleeve 46 rather than leaked to the environment through the priming opening 48 due to the high surface tension and high viscosity of the blood.

In some embodiments, connecting the blood collection tube 50 to the holder 34 may squeeze the elastomeric sleeve 46 to firmly seal the elastomeric sleeve 46 at the priming opening 48. In some embodiments, the blood collection device 12 may then begin to take the blood sample automatically. The blood collection device 12 may automatically stop taking the blood sample upon disconnecting the blood collection tube 50 from the holder 34. Similarly, some embodiments of the blood collection device 12 may automatically conduct an air priming procedure prior to sample collection and may automatically stop the air priming procedure when the blood collection tube 50 is attached to the blood collection device 12.

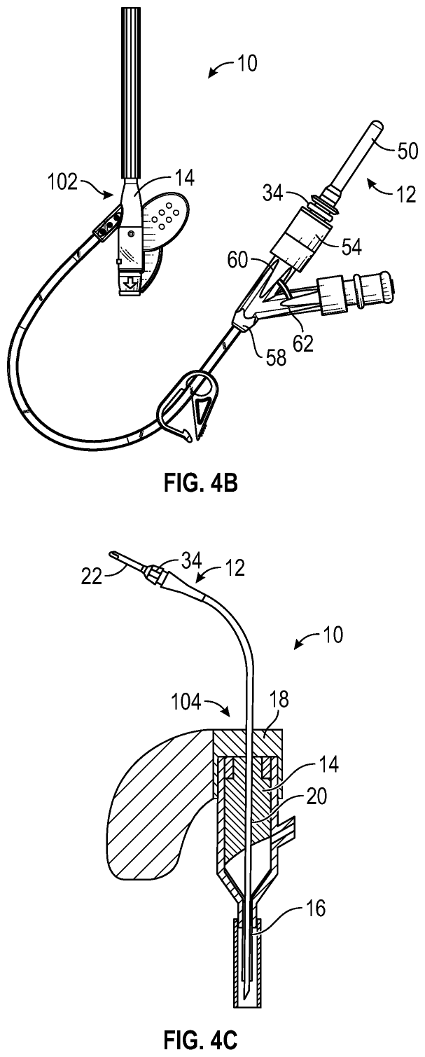

Referring now to FIGS. 4A-4C, in some embodiments the holder 34 may be configured to couple to the blood collection tube 50 such that the sharp proximal end 78 of the cannula 22 and the elastomeric sleeve 46 may extend through a distal end of the blood collection tube 50. Some embodiments of the blood collection device 12 may be used independently or may be used in connection with an existing catheter system or blood collection set 102. As shown in FIG. 4A, some embodiments of the blood collection device 12 may attach to a blood collection system 10 including a peripheral IV catheter (“PIVC”), for example. As shown in FIG. 4B, some embodiments of the holder 34 may be configured to couple to a needleless adapter 54. As shown in FIG. 4C, some embodiments of the holder 34 may be configured to couple to a proximal end of a catheter adapter 104.

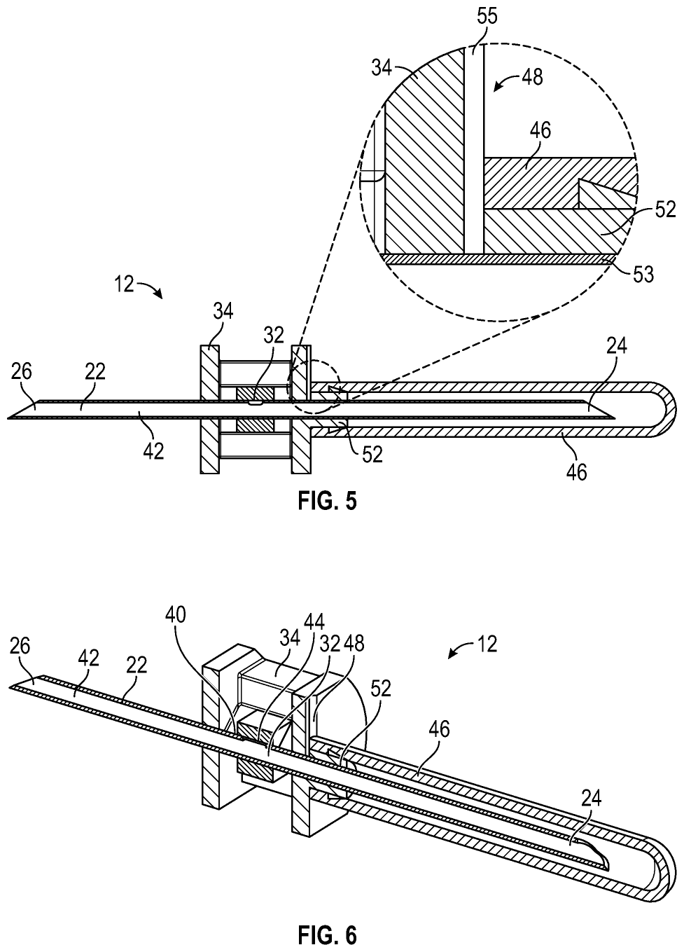

Referring to FIGS. 5 and 6, in some embodiments, the holder 34 may include a projection 52 extending from the proximal end 36 of the holder 34 along the longitudinal axis 30. Some embodiments of the projection 52 may include at least a portion of the channel 40. In some embodiments, the shaft 42 of the cannula 22 may be retained by the channel 40 such that the projection 52 supports the proximal end 24 of the cannula 22 as it extends into the elastomeric sleeve 46.

In some embodiments, the projection 52 may include one or more priming openings 48. As shown in the enlarged view of FIG. 5, for example, a clearance opening 53 may extend through the projection 52 along the longitudinal axis 30. In this manner, some embodiments of the projection 52 may provide clearance space between an outer surface of the cannula 22 and an inner surface of the projection 52, thereby enabling the cannula 22 to be manipulated with respect to the projection 52.

As shown in FIG. 6, some embodiments of the cannula 22 may include a slot 32 to provide unobstructed visualization of a small volume of blood through the cannula 22. In some embodiments, visualization of such blood flashback may provide vein confirmation.

Some embodiments of the holder 34 may include a visual check window 44 configured to align with the slot 32 when the holder 34 retains the shaft 42 of the cannula 22. Some embodiments of the visual check window 44 may include an aperture or slot disposed in the holder 34 and positioned to correspond to the slot 32 in the cannula 22. In some embodiments, the visual check window 44 may include an opening having a size and shape at least as large as the slot 32 such that blood flashback may be visualized therethrough. In some embodiments, the visual check window 44 may include graduated dimensions that increase from an inside surface of the holder 34 adjacent to the slot 32 toward an exterior surface of the holder 34. Some embodiments of the visual check window 44 may include a circular shape, a rectangular shape, a polygonal shape, or any other suitable shape and/or cross-sectional shape.

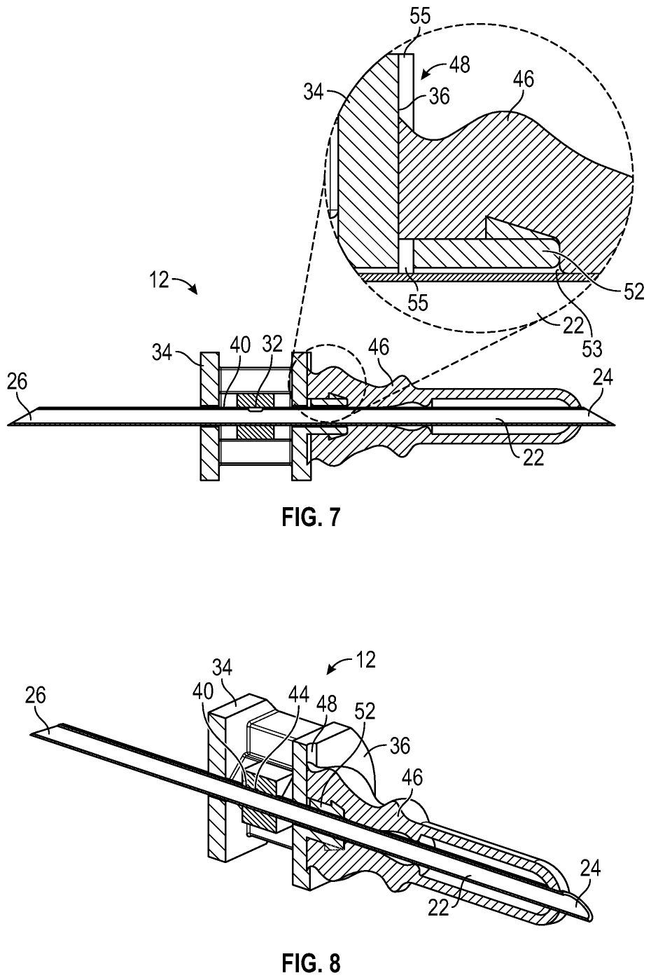

Referring now to FIGS. 7 and 8, in some embodiments, the projection 52 may include one or more priming openings 48 between the proximal end 36 of the holder 34 and the projection 52. In some embodiments, the priming opening 48 may include a priming slot 55 disposed along the proximal end 36 of the holder 34 in a transverse direction relative to the longitudinal axis 30. In some embodiments, the priming opening 48 may further include a clearance opening 53 integrated through the holder 34 and projection 52 along the longitudinal axis 30 and between the channel 40 and the cannula 22.

Some embodiments of the priming slot 55 may be selectively blocked by the elastomeric sleeve 46 upon application of an external force, such as a force applied by the blood collection tube 50 upon being attached to the proximal end of the holder 34. In some embodiments, the elastomeric sleeve 46 may deform to passively block the priming slot 55 or other priming opening 48. In some embodiments, deformation of the elastomeric sleeve 46 may cause the proximal end 24 of the cannula 22 to extend through a proximal end of the elastomeric sleeve 46.

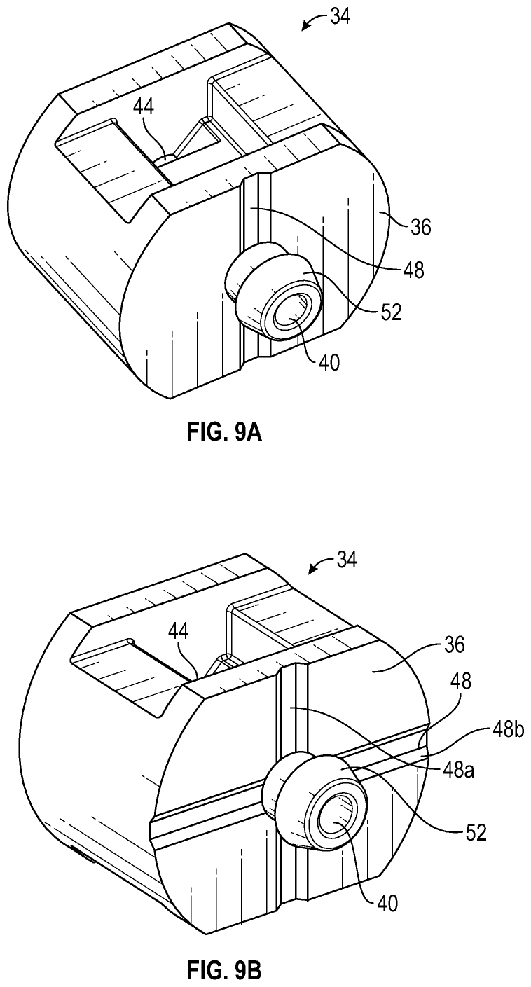

FIGS. 9A-B, 10A-B, 11A-B, 12A-B, and 13A-B illustrate various embodiments of priming openings 48 integrated into various embodiments of holders 34 and projections 52. As shown in FIGS. 9A and 9B, for example, some embodiments of a priming opening 48 may include one or more priming slots 55 integrated into an exterior surface of the proximal end 36 of the holder 34. Some embodiments may include a single priming slot 55 disposed in a transverse direction relative to the longitudinal axis 30, as shown in FIG. 9A. Some embodiments may include two priming slots 55 disposed in opposite directions relative to the longitudinal axis 30, as shown in FIG. 9B. In some embodiments, the projection 52 may be coupled to or integrated with the proximal end 36 of the holder 34 such that the projection 52 may be superimposed onto the one or more priming slots 55. In some embodiments, the channel 40 may extend through a center of the projection 52 and holder 34 to retain the cannula 22 therein.

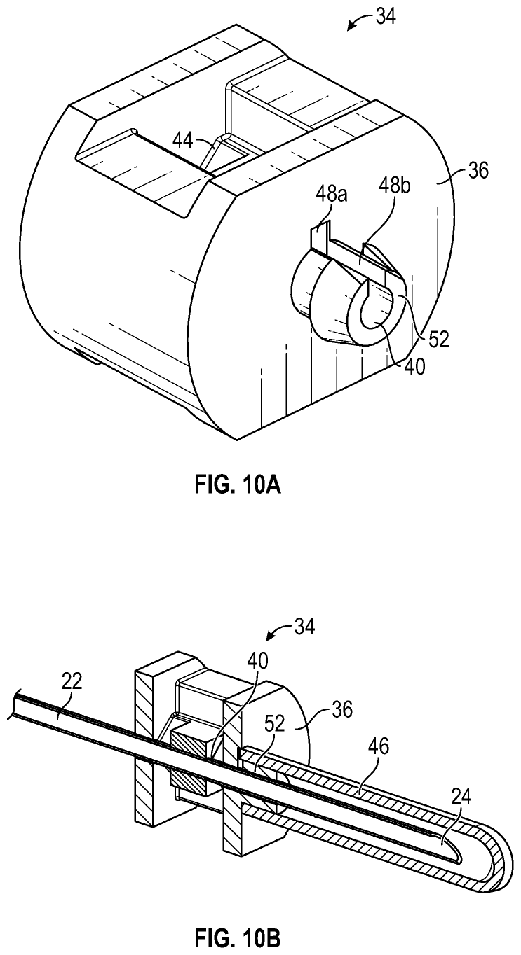

As shown in FIGS. 10A and 10B, some embodiments of a projection 52 may include a priming opening 48 integrated into a side wall thereof such that the channel 40 is open on at least one side. In some embodiments, the priming opening 48 may be further integrated into at least a portion of the proximal end 36 of the holder 34.

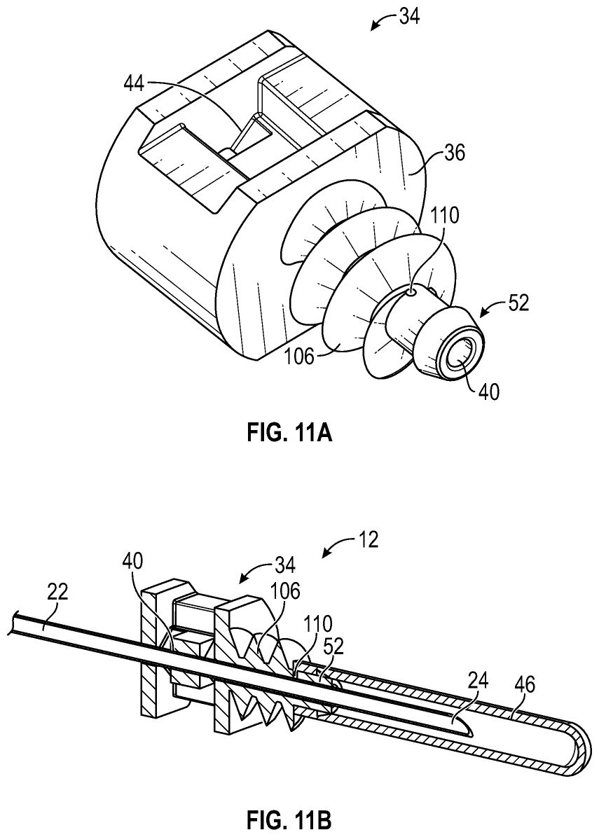

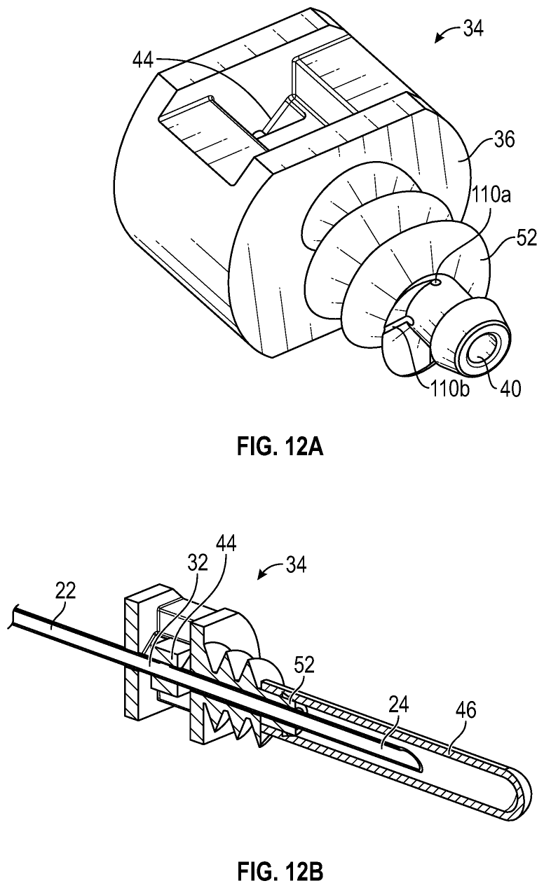

As shown in FIGS. 11A and 11B, some embodiments of the holder 34 may include an elongate portion 108 having the projection 52 extending therefrom along the longitudinal axis 30. In some embodiments, the elongate portion 108 may include one or more threads 106 or other suitable engagement mechanism. Some embodiments of the elongate portion 108 may include the priming opening 48. As shown, the priming opening 48 may include a single prime hole 110 integrated into the elongate portion 108 near the interface between the proximal end of the elongate portion 108 and the distal end of the elastomeric sleeve 46. In other embodiments, as shown in FIGS. 12A and 12B, the elongate portion 108 may include multiple prime holes 110 integrated into the surface and/or threads 106 thereof.

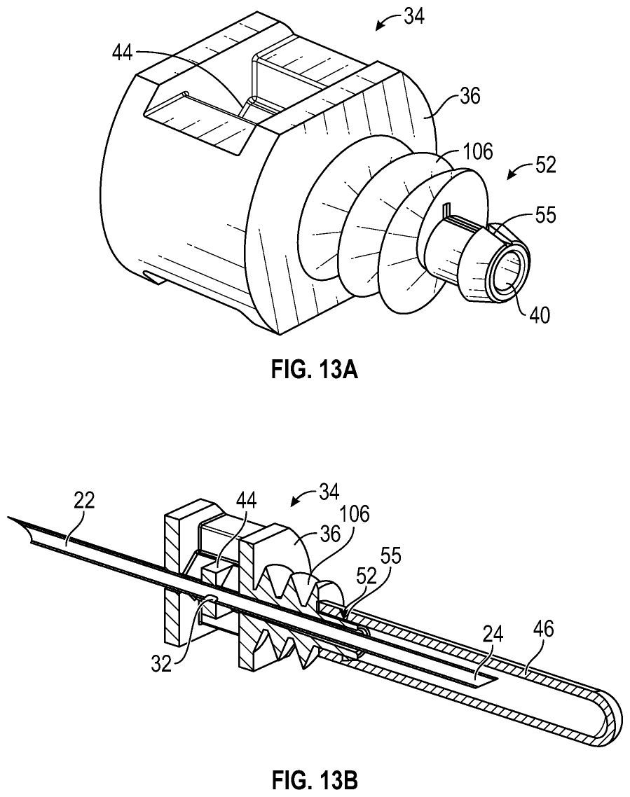

Referring now to FIGS. 13A and 13B, in some embodiments, the priming opening 48 may include a priming slot 55 integrated into the projection 52 and extending into at least a portion of the elongate portion 108 and/or threads 106. In any case, the priming slot 55 may be blocked and/or sealed by the elastomeric sleeve 46 upon an external application of force to deform the elastomeric sleeve 46.

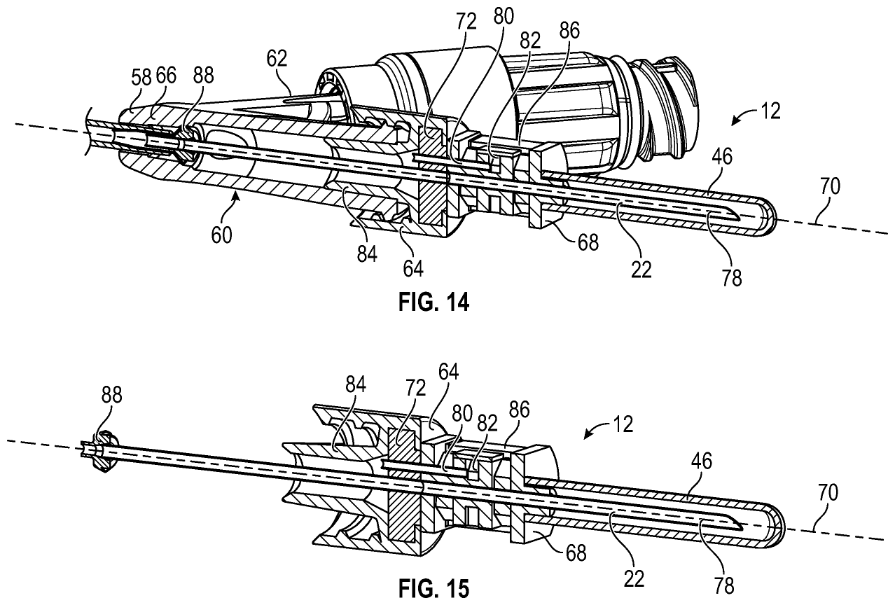

Referring now to FIGS. 14 and 15, some embodiments of the blood collection device 12 may include a body 64 having a distal end 66, a proximal end 68, and a longitudinal axis 70 extending therebetween. In some embodiments, an exterior surface of the body 64 may provide a contact interface 56 for a user's grip. Some embodiments of the contact interface 56 may include ridges, bumps, mesh, or another suitable texture or structure to provide a solid grip. In some embodiments, a septum 72 may be disposed within the body 64.

In some embodiments, a cannula 22 having a distal end 76 and a sharp proximal end 78 may extend through the septum 72. In some embodiments, the cannula 22 may be a steel cannula 22 or may include any other suitable material. In some embodiments, an elastomeric sleeve 46 may be coupled to the body 64 to cover the sharp proximal end 78 of the cannula 22. Some embodiments of the elastomeric sleeve 46 may be liquid impermeable and biocompatible. For example, in some embodiments, the elastomeric sleeve 46 may include rubber or another suitable material. In some embodiments, the elastomeric sleeve 46 may automatically seal an air priming pathway in response to a blood collection tube 50 being coupled to the body 64.

In some embodiments, the blood collection device 12 may be coupled to a port of an adapter 14. As shown in FIG. 14, for example, some embodiments of the adapter 14 may include a first proximal port 60, a second proximal port 62, and a distal port 58. In some embodiments, the blood collection device 12 may be coupled to the first proximal port 60 via a needleless adapter 54 such as a Luer adapter. For example, as shown, some embodiments of the blood collection device 12 may include a male Luer adapter 84 configured to connect to the first proximal port 60. In some embodiments, the distal end 66 of the body 64 of the blood collection device 12 may include the male Luer adapter 84 and the cannula 22 may extend through the proximal end 68 of the body 64. In some embodiments, the proximal end 68 of the body 64 may be configured to couple to a blood collection tube 50, such as a VACUTAINER™. In some embodiments, the sharp proximal end 78 of the cannula 22 may extend into the blood collection tube 50 to provide a pathway for blood sampling.

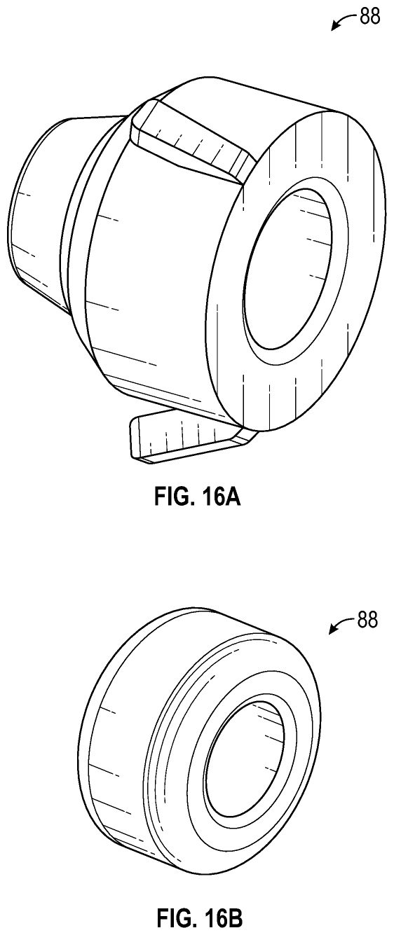

In some embodiments, the air priming pathway of the blood collection device 12 may be separate from a blood collection pathway to prevent blood from contaminating the first proximal port 60. In some embodiments, as shown in FIGS. 16A and 16B for example, a seal ring 88 may be disposed within the first proximal port 60 and the cannula 22 may extend therethrough. Some embodiments of the seal ring 88, such as that shown in FIG. 16A, may be disposed adjacent to the septum 72. Other embodiments of the seal ring 88, such as that shown in FIG. 16B, may be disposed at a distal end 66 of the first proximal port 60. In some embodiments, the seal ring 88 may isolate blood within the cannula 22 and prevent blood from contaminating the inner cavity of the adapter 14.

In some embodiments, the body 64 may include at least a portion of the air priming pathway to prime air out of the adapter 14. For example, some embodiments of the body 64 may include an air pipe 80 and an air membrane 82. In some embodiments, the air pipe 80 may extend through the septum 72. In some embodiments, the air pipe 80 may puncture through the septum 72. Some embodiments of the septum 72 may self-seal upon withdrawal of the air pipe 80 and/or cannula 22. In some embodiments, the body 64 and septum 72 of the blood collection device 12 may be used as a needleless adapter 54 upon withdrawing the air pipe 80 and cannula 22 therefrom.

Some embodiments of the air membrane 82 may be disposed proximal to the air pipe 80. In some embodiments, the body 64 may include an extension 86 between the septum 72 and the elastomeric sleeve 46 that is configured to be held by a user. Some embodiments of the air membrane 82 may be disposed within the extension 86. In some embodiments, the air priming pathway, including the air pipe 80 and the air membrane 82, may be automatically sealed in response to the blood collection tube 50 being coupled to the body 64.

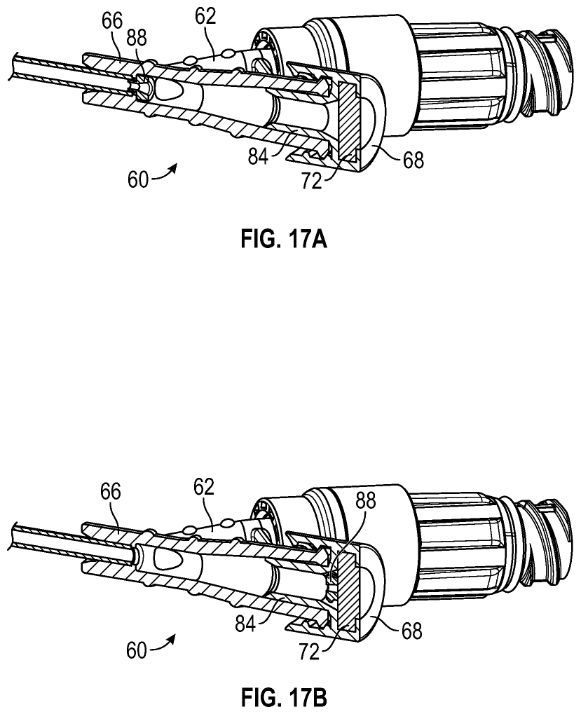

Referring now to FIGS. 17A and 17B, in some embodiments, at least a portion of the blood collection device 12 may be disposable while remaining components of the blood collection device 12 may be reusable, thereby promoting procedure efficiencies and future blood sampling procedures. As shown in FIGS. 17A and 17B, in some embodiments, the cannula 22, the body 64, the air pipe 80 and air membrane 82, and the elastomeric sleeve 46 may be disposed of for infusion or indwelling, for example, while the seal ring 88, the male luer adapter 84, and the septum 72 may remain coupled to the first proximal port 60 of the adapter 14 and reused. As previously discussed, the seal ring 88 may be seated at the distal end 66 or proximal end 68 of the body 64 depending on its purpose and/or manufacturing process.

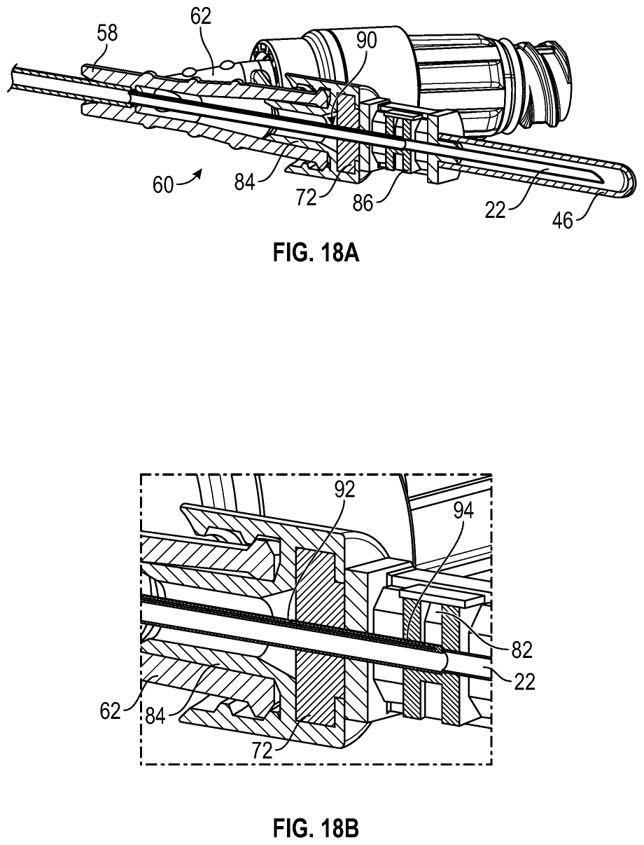

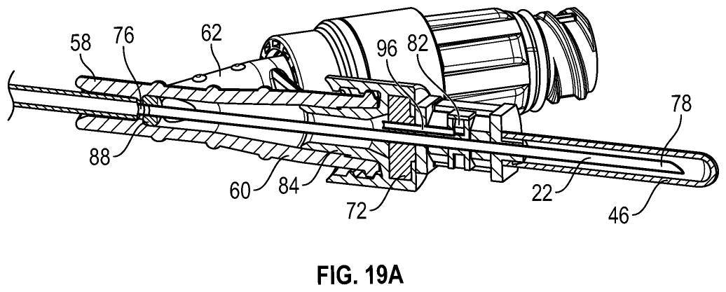

Referring now to FIGS. 18A and 18B, in some embodiments, the air pipe 80 may include multiple channels 90. In some embodiments, some of the channels 90 may be used for blood sampling while other channels 90 may be used for air priming. As shown in FIG. 18B, for example, in some embodiments a first channel 92 may be used for blood collection and a second channel 94 may be used for air priming. In some embodiments, the second channel 94 may be sealed at both ends and may include one or more apertures to provide air priming.

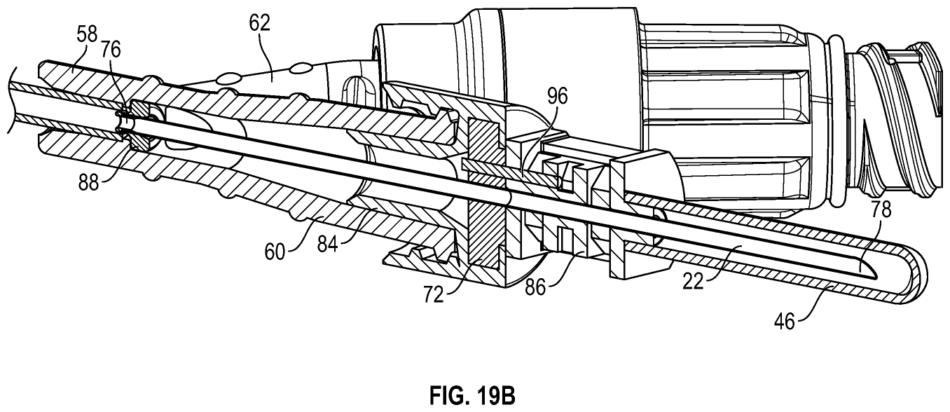

Referring now to FIGS. 19A and 19B, some embodiments of the air priming pathway may include a porous air rod 96 instead of the air pipe 80 and the air membrane 82. Some embodiments of the air rod 96 may include a porous material that is air permeable and liquid impermeable. In this manner, some embodiments of the air rod 96 may be configured to prevent liquid from penetrating the air rod 96 while allowing a free flow of air therethrough.

In some embodiments, the air rod 96 may be integrated with the body 64 of the blood collection device 12. In other embodiments, the air rod 96 may be removed from the body 64 of the blood collection device 12 and/or the septum 72 after performance of a blood sampling procedure.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A blood collection device to prime a blood flow path, comprising:
a cannula comprising a proximal end, a distal end, and a lumen extending along a longitudinal axis therebetween, the cannula comprising a slot to enable a user to visualize a blood flow path through the cannula;
a holder comprising a proximal end, a distal end, and a channel extending therebetween, the channel configured to align with the longitudinal axis to retain a shaft of the cannula, the holder comprising a visual check window corresponding to the slot of the cannula; and
an elastomeric sleeve coupled to the holder and enclosing the proximal end of the cannula, wherein a priming opening is disposed between the elastomeric sleeve and the holder to prime the blood flow path.

2. The blood collection device of claim 1, wherein the priming opening is automatically sealed in response to a blood collection tube being coupled to the holder.

3. The blood collection device of claim 2, wherein the elastomeric sleeve automatically seals the priming opening.

4. The blood collection device of claim 2, wherein the holder is configured to couple to the blood collection tube such that the proximal end of the cannula and the elastomeric sleeve extend through a distal end of the blood collection tube.

5. The blood collection device of claim 1, wherein the holder further comprises a projection extending from the proximal end of the holder along the longitudinal axis.

6. The blood collection device of claim 5, wherein the projection comprises at least a portion of the channel.

7. The blood collection device of claim 5, wherein the projection comprises the priming opening.

8. The blood collection device of claim 1, wherein the holder is configured to couple to a needleless connector.

9. The blood collection device of claim 1, wherein the holder comprises a contact interface for physical manipulation of the cannula.

10. A blood collection system, comprising:
an adapter comprising a distal port, a first proximal port, and a second proximal port;
a blood collection device coupled to the first proximal port, the blood collection device comprising:
a body comprising a distal end, a proximal end, and longitudinal axis extending therebetween;
a septum disposed within the body;
a cannula comprising a distal end and a sharp proximal end, wherein the cannula extends through the septum;
an elastomeric sleeve coupled to the body and covering the sharp proximal end of the cannula;
an air pipe extending through the septum; and
an air membrane disposed proximal to the air pipe;
wherein an air priming pathway comprises the air pipe and the air membrane, and wherein a blood collection pathway separate from the air priming pathway comprises the cannula.

11. The blood collection system of claim 10, wherein the distal end of the body comprises a male luer adapter, and wherein the cannula extends through the proximal end of the body.

12. The blood collection system of claim 10, wherein the body comprises an extension between the septum and the elastomeric sleeve configured to be held by a user, wherein the air membrane is disposed within the extension.

13. The blood collection system of claim 10, wherein the elastomeric sleeve automatically seals the air priming pathway in response to a blood collection tube being coupled to the body.

14. The blood collection system of claim 10, further comprising a seal ring disposed within the first proximal port such that the cannula extends therethrough.

15. The blood collection system of claim 14, wherein the seal ring is disposed at a distal end of the first proximal port.

16. The blood collection system of claim 14, wherein the seal ring is disposed adjacent to the septum.

17. The blood collection system of claim 10, wherein the air pipe comprises a plurality of channels.

18. The blood collection system of claim 10, wherein the blood collection pathway comprises a first channel of the plurality of channels, and wherein the air priming pathway comprises a second channel of the plurality of channels.

19. The blood collection system of claim 10, wherein the air priming pathway comprises an air rod to replace the air pipe and the air membrane, the air rod comprising a porous material to allow air therethrough and block liquid.

20. The blood collection system of claim 19, wherein the air rod is integrated with the body.
